(19) United States
(12) Patent Application Publication
(10) Pub. No.: US 2004/0022836 A1
(43) Pub. Date: Feb. 5, 2004

(54) Transdermal therapeutic systems comprising photosensitive active substances

(76) Inventors: Anja Degen, Wehr (DE); Frank Theobald, Bad Breisig (DE)

Correspondence Address: BIRCH STEWART KOLASCH & BIRCH, PO BOX 747, FALLS CHURCH, VA, 22040-0747, US

(21) Appl. No.: 10/415,144

(22) PCT Filed: Oct. 18, 2001
(86) PCT No.: PCT/EP01/12068

(30) Foreign Application Priority Data
Oct. 27, 2000 (DE) 100 53 375.2

(51) Int. Cl. A61K 9/70

(52) U.S. Cl. 424/449

(57) ABSTRACT

Transdermal therapeutic systems (TTS) whose structure comprises a polymer matrix containing active substances, and a backing layer, and which contain at least one light-sensitive active substance are characterized in that the said TTS contain at least one colourless substance which absorbs light in the UV region, does not have any inherent pharmacological action, is dispersed or dissolved in the polymer matrix of the TTS, and/or is homogeneously distributed in the backing layer of the TTS.

The present invention relates to transdermal therapeutic systems (TTS) containing light-sensitive active substances. More particularly, the invention relates to TTS which are of a transparent or colourless-transparent appearance.

Various pharmaceutically active substances, e.g. nicotine or nifedipine, possess a high sensitivity to light. In pharmaceutical preparations containing such light-sensitive active substances, photochemical degradation of the active substance under the action of daylight or sunlight, and consequently a significant decrease in the active substance content, may occur if during storage of the preparations up to the time of application, or during the duration of application, the active substances are not protected against admission of light.

With the classical application forms such as, for example, orally, parenterally or conjuntivally applied administration forms, a sufficient resistance to the action of daylight is mostly achieved already by selecting a suitable primary or secondary package preventing the admission of light to the active substance. Since as a rule there is only a short period of time between taking the administration form out of the package and administering the same, a degradation of the active substance as a consequence of action of light is largely excluded in these administration forms. If a longer duration of application is required, such as when using infusion solutions, application is mostly stationary, it being possible to use dyed or secondary-packed infusion bottles in order to protect the light-sensitive active substances against degradation. The above-mentioned measures are as a rule sufficient to ensure the stability of the active substance to be applied, during storage and/or during the time span of application.

However, these classical application forms differ from transdermal therapeutic systems (TTS). The latter represent systems loaded with active substance, with the active substances being contained in self-adhesive or non-self-adhesive polymers of differing chemical composition. The active substances contained therein are continuously delivered to the patient's skin, i.e. a TTS is applied to the skin and remains there for a prolonged period of time, for example for several hours up to several days.

As a result, the active substance is even during the duration of application exposed to a greater or lesser degree to the daylight, depending on the respective application site, and can experience a significant, not negligible loss of active substance during the time of application. In the extreme case, for example in the case of particularly light-sensitive active substances, this can lead to the active substance delivery falling short of the therapeutically required amount, thus jeopardizing the therapeutic result.

In the TTS available on the market which contain light-sensitive active substances, the problem is generally solved by using an aluminised or coated cover film. This film forms the backing layer of the system and outwardly covers the active substance-containing matrix so that the admission of daylight to the active substance-containing matrix is minimized, thereby protecting the active substance from being degraded by sun light.

DE-A1-199 12 623, for instance, proposes that in order to improve the stability of light-sensitive TTS, the TTS be equipped with dyed plastics films as cover films.

In some cases, however, this method of achieving a protection from light by way of employing aluminized, coated or dyed covering films may be not desired, or lead to problems or drawbacks.

Dying or aluminising of highly flexible plastic films is generally difficult and does not offer reliable light protection since as a consequence of the film becoming stretched there may occur cracks in the dye layer or in the aluminizing layer which allow partial entry of light into the active substance-containing polymer matrix and which can thus lead to the degradation of the active substance in the matrix.

As an alternative to dyed or aluminized cover films there are flexible, dyed fabrics, which can be highly elastic in some cases. These do, however, have the disadvantage of as a rule not being suited for application over several days since they are not able to withstand the environmental influences occurring in this case, especially when a person is taking a shower, is sweating or visiting a sauna etc.

Aluminized, coated or dyed covering films additionally have the disadvantage of being optically very conspicuous and thus can lead to a patient being stigmatized. The patient, when wearing a TTS provided with such cover layers, can become recognizable as a “sick” person, which can lead to social isolation and, on the part of the patient, to insufficient compliance or acceptance.

It was thus the object of the present invention to provide transdermally applicable pharmaceutical preparations containing light-sensitive active substances wherein the resistance to the influence of light is increased without the occurrence of the aforementioned drawbacks.

According to the invention, this object is achieved in transdermal therapeutic systems (TTS) whose structure comprises a polymer matrix containing active substance, and a backing layer, and which have a content of at least one light-sensitive active substance, in that at least one colourless substance absorbing light in the UV range is homogeneously distributed in the active substance-containing matrix, e.g. in dissolved or dispersed form, and/or in that such a substance is homogeneously distributed in the backing layer (cover film) thereof. The substance absorbing light in the UV region has no inherent pharmacological action, i.e. it is itself not therapeutically active.

Light-sensitive active substances are, for example, nicotine, or active substances from the group of dihydropyridine derivatives, e.g. nifedipine or lacidipine, or gestagens, vitamin B 12, and antibiotics, as well as salts of such light-sensitive substances.

Due to the presence of a UV-absorbing, colourless substance it is made possible to produce TTS which have a transparent backing layer and/or a transparent active substance matrix and which nevertheless ensure the protection of the light-sensitive active substances against light-induced degradation. It is of particular advantage that in this way it is possible to produce TTS which are completely transparent and therefore are hardly conspicuous when being worn on the skin. This is the case, in particular, where the TTS, according to a further, preferred embodiment, is configured to be transparent and colourless, that is, where both the backing layer (cover film) and the polymer matrix and, possibly, further layers are transparent and colourless.

As materials for the covering film of the inventive TTS, transparent films of polyester, polyethylene, polypropylene, polyurethane, ethylene vinyl acetate, polyethylene terephthalate (PET) or mixtures of such polymers are used with preference.

The active substance-containing polymer matrix of the TTS according to the invention may be single- or multi-layered; preferably, it has pressure sensitive adhesive properties. It is fixedly connected with the backing layer (cover layer) or forms a laminate therewith. The skin-facing, pressure sensitive adhesive surface of the polymer matrix is commonly covered by a peelable protective layer or protective film which is removed prior to application. This protective film, too, can be adapted to be impermeable to light.

As base materials for the polymer matrix of the TTS according to the invention, polyacrylates, polyisobutylenes, polydimethyl siloxanes, styrene-isoprene block copolymers or isoprene polymers with or without synthetic or partial-synthetic polymers are used with preference.

In any case, on account of the presence of a substance absorbing light in the UV region, also called UV-absorber or UV-blocker, the light-sensitive active substance is protected against photochemical degradation. The term “UV region” is understood to mean the region of the electromagnetic spectrum which lies between 100 nm and 400 nm. For the intended purpose, it is in most cases sufficient if the UV-absorbing substances absorb light in the region of 250 nm to 400 nm. Preferably, the UV-absorbing substances used absorb light in the UV-A region and/or in the UV-B region (so-called UV-A absorbers or UV-B absorbers).

With respect to selecting the UV absorbers it is preferred for the UV absorber to have an absorption maximum which lies within the wavelength region which causes the degradation of the active substance used.

To achieve a protection from photochemical degradation over a broader UV spectral region, it is advantageous if the inventive TTS contain a combination of at least two substances absorbing light in the UV region which have different absorption maxima.

In principle, UV absorbers are employed with preference which have already been proven to be harmless when used in cosmetic products, or whose application on the skin is toxicologically harmless.

The entire constituent amount of the added UV absorbers preferably lies in the region of 1-20%-wt., especially preferred in the region of 5-10%-wt., in each case relative to one TTS.

The substance(s) absorbing light in the UV region is/are preferably selected from the group comprising p-amino-benzoic acid and aminobenzoic acid derivatives, preferably 4-dimethylaminobenzoic acid-2-ethylhexyl ester, 4-bis(polyethoxyl)aminobenzoic acid-polyethoxyethyl ester, as well as cinnamic acid and its derivatives, preferably 4-methoxycinnamic acid isoamyl ester, 4-methoxycinnamic acid-2-ethylhexyl ester, as well as 3-benzylidene bornan-2-one and benzylidene bornan-2-one derivatives, preferably 3-(4′)methylbenzylidene-bornan-2-one, 3-(4-sulfo)benzylidene bornan-2-one, 3-(4′-trimethylammonium) benzylidene bornan-2-one-methyl sulphate, as well as salicylic acid derivatives, preferably 4-isopropylbenzyl salicylate, salicylic acid-2-ethylhexyl ester, 3,3,5-trimethyl-cyclohexyl salicylate, as well as 2,4,6-trianiline-p-(carbo-2′-ethyl-hexyl-1′-oxy)-1,3,5-triazine, 3-imidazol-4-yl-acrylic acid and its esters, 2-phenylene benzimidazol-5-sulfonic acid and their K-, Na- and triethanol amine (=TEA) salts, 2-cyano-3,3-diphenyl-acrylic acid, terephthaloylidene-dicamphor-sulfonic acid, butylmethoxydibenzoylmethane, as well as benzophenones or benzophenone derivatives, preferably benzophenone-3, benzophenone-4.

The invention and its advantageous properties will be illustrated further by way of the following example.

### EXAMPLE

Two formulations (A, B) of a light-sensitive active substance from the group of gestagens were prepared which differed in their composition insofar as one formulation (B) contained 10%-wt. of a UV absorber whereas the other formulation (A) contained no UV absorber while otherwise being of the same composition. Both pressure sensitive adhesive, active substance-containing laminates were provided with a transparent cover film of PET, which yielded a “TTS”.

The composition of the formulation (B) is as follows: (all indications in percent by weight)

| | |
|---|---|
| 2.0% | gestagen |
| 87.6% | acrylate polymer |
| 0.4% | cross-linking agent |
| 10.0% | Eusolex ® 6300 |

Eusolex® 6300 (Merck, Darmstadt) is an oil-soluble UV-B absorber (3-(4-methylenebenzylidene)-camphor).

To test the light-protection action, both TTS formulations, covered with PET films, were irradiated according to the ICH Guideline “Note for guidance on the photostability testing of new active substances and medicinal products” (CPMP/ICH/279/95). Irradiation time was 7 h; as the radiation source, a xenon lamp was used. Due to its construction, the light source utilized generates an emission of light comparable to the D65/ID65 emission standard. Subsequently, the active substance content in the TTS was determined.

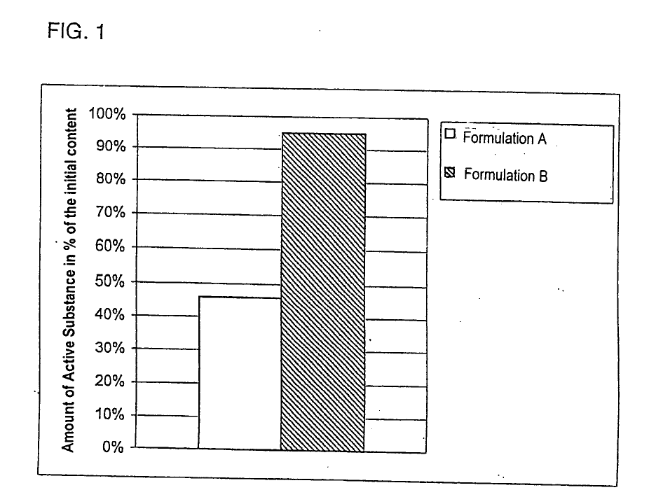

The results are graphically illustrated in FIG. 1.

It turned out that in the case of the TTS formulation (B), which contained UV absorbers, about 95% of the light-sensitive active substance employed could be found again whereas in the TTS formulation (A), which contained no UV absorber, only 46% of the originally present amount of active substance could be detected after irradiation. This shows that the addition of UV absorbers as proposed according to the invention prevents the photochemical degradation of active substances and therefore allows producing TTS which have a content of light-sensitive active substances as transparent TTS, and thereby improving their acceptance or compliance.

1. Transdermal therapeutic systems (TTS) whose structure comprises a polymer matrix containing active substance(s), and a backing layer, and which contain at least one light-sensitive active substance, characterized in that the said TTS contain at least one colourless substance which absorbs light in the UV region, does not have any inherent pharmacological action, is dispersed or dissolved in the polymer matrix of the TTS, and/or is homogeneously distributed in the backing layer of the TTS.

2. TTS according to claim 1, characterized in that they are equipped with a transparent backing layer whereby a transparent film of polyester, polyethylene, polypropylene, polyurethane, ethylene vinyl acetate, or mixtures of such polymers, are preferably used as the backing layer.

3. TTS according to claim 1 or 2, characterized in that the backing layer is colourless-transparent.

4. TTS according to any one of claims 1 to 3, characterized in that the active substance-containing reservoir is transparent, preferably colourless-transparent.

5. TTS according to any one of claims 1 to 4, characterized in that it is overall transparent, preferably transparent and colourless.

6. TTS according to one or more of the preceding claims, characterized in that the polymer matrix is essentially made of polymers selected from the group comprising polyacrylates, polyisobutylenes, polydimethyl siloxanes, styrene-isoprene block copolymers, isoprene polymers with or without synthetic or partially synthetic polymers.

7. TTS according to one or more of the preceding claims, characterized in that they contain the UV-absorbing substance(s) in a portion of 1-20%-wt., preferably 5-10%-wt.

8. TTS according to one or more of the preceding claims, characterized in that the substance(s) which absorb light in the UV region do so in the UV-A and/or the UV-B region, preferably in the wavelength region of 250-400 nm.

9. TTS according to one or more of the preceding claims, characterized in that the substance(s) absorbing light in the UV region is/are selected from the group comprising p-aminobenzoic acid and aminobenzoic acid derivatives, preferably 4-dimethylaminobenzoic acid-2-ethylhexyl ester, 4-bis(polyethoxyl)aminobenzoic acid-polyethoxyethyl ester, as well as cinnamic acid and its derivatives, preferably 4-methoxycinnamic acid isoamyl ester, 4-methoxycinnamic acid-2-ethylhexyl ester, as well as 3-benzylidene bornan-2-one and benzylidene bornan-2-one derivatives, preferably 3-(4′)methylbenzylidene-bornan-2-one, 3-(4-sulfo)benzylidene bornan-2-on, 3-(4′-trimethylammonium)benzylidene bornan-2-one-methyl sulphate, as well as salicylic acid derivatives, preferably 4-isopropylbenzyl salicylate, salicylic acid-2-ethylhexyl ester, 3,3,5-trimethyl-cyclohexyl salicylate, as well as 2,4,6-trianiline-p-(carbo-2′-ethyl-hexyl-1′-oxy)-1,3,5-triazine, 3-imidazol-4-yl-acrylic acid and its esters, 2-phenylene benzimidazol-5-sulfonic acid and its K-, Na- and triethanol amine (=TEA) salts, 2-cyano-3,3-diphenyl-acrylic acid, terephthaloylidene dicamphor sulfonic acid, butylmethoxydibenzoylmethane, as well as benzophenones or benzophenone derivatives, preferably benzophenone-3, benzophenone-4.

10. TTS according to one or more of the preceding claims, characterized in that the absorption maximum of the substances absorbing light in the UV region is within that wavelength region which causes the degradation of the active substance used.

11. TTS according to one or more of the preceding claims, characterized in that they contain a combination of at least two substances absorbing light in the UV region which have different absorption maxima.
